(19) United States
(12) Patent Application Publication
(10) Pub. No.: US 2025/0205634 A1
(43) Pub. Date: Jun. 26, 2025

(54) CARBON DIOXIDE RECOVERY APPARATUS AND CARBON DIOXIDE RECOVERY METHOD

(71) Applicant: TOYOTA JIDOSHA KABUSHIKI KAISHA, Toyota-shi (JP)

(72) Inventors: Takahiro SADAMITSU, Miyoshi-shi (JP); Masaki Watanabe, Sunto-gun (JP); Mitsuru Sakano, Toyota-shi (JP); Satsuki Osada, Susono-shi (JP)

(73) Assignee: TOYOTA JIDOSHA KABUSHIKI KAISHA, Toyota-shi (JP)

(21) Appl. No.: 18/933,513

(22) Filed: Oct. 31, 2024

(30) Foreign Application Priority Data
Dec. 20, 2023 (JP) 2023-214991

(51) Int. Cl. B01D 53/04

(52) U.S. Cl. CPC B01D 53/0454; B01D 2253/202; B01D 2257/504; B01D 2257/80

(57) ABSTRACT

A carbon dioxide recovery apparatus and a carbon dioxide recovery method by which energy consumption can be reduced so as to improve CO₂ recovery efficiency is provided. A carbon dioxide recovery apparatus includes: an adsorbent configured to adsorb CO₂ and H₂O; a reaction vessel inside which the adsorbent is disposed; H₂O value detection means for detecting an H₂O value that is a value related to an amount of H₂O adsorbed into the adsorbent; and flow rate adjustment means for adjusting a flow rate of gas flowing into the reaction vessel based on the H₂O value detected by the H₂O value detection means.

### CROSS REFERENCE TO RELATED APPLICATIONS

This application is based upon and claims the benefit of priority from Japanese patent application No. 2023-214991, filed on Dec. 20, 2023, the disclosure of which is incorporated herein in its entirety by reference.

### BACKGROUND

The present disclosure relates to a carbon dioxide recovery apparatus and a carbon dioxide recovery method for recovering carbon dioxide.

A carbon dioxide recovery apparatus including an adsorbent that adsorbs CO₂ and H₂O, a reaction vessel in which the adsorbent is disposed, and an H₂O concentration adjustment device that adjusts the concentration of H₂O in gas and allows the adjusted gas to flow into the reaction vessel is known (see, for example, Patent Literature 1).
Patent Literature 1: Japanese Unexamined Patent Application Publication No. 2019-034307

### SUMMARY

In the above carbon dioxide recovery apparatus, the concentration of H₂O in gas is adjusted using the H₂O concentration adjustment device. Therefore, energy is required to operate the H₂O concentration adjustment device, and thus energy consumption may be increased so as to decrease CO₂ recovery efficiency.

The present disclosure has been made in order to solve the above-described problem and a main object thereof is to provide a carbon dioxide recovery apparatus and a carbon dioxide recovery method by which energy consumption can be reduced so as to improve CO₂ recovery efficiency.

In order to achieve the above-described object, one aspect according to the present disclosure is a carbon dioxide recovery apparatus including:
an adsorbent configured to adsorb CO₂ and H₂O;
a reaction vessel inside which the adsorbent is disposed;
H₂O value detection means for detecting an H₂O value that is a value related to an amount of H₂O adsorbed into the adsorbent; and
flow rate adjustment means for adjusting a flow rate of gas flowing into the reaction vessel based on the H₂O value detected by the H₂O value detection means.

In the above aspect, the flow rate adjustment means may increase the flow rate of gas flowing into the reaction vessel when the H₂O value detected by the H₂O value detection means is greater than a predetermined value.

In the above aspect, the flow rate adjustment means may reduce the flow rate of gas flowing into the reaction vessel when the H₂O value detected by the H₂O value detection means is less than a predetermined value.

In the above aspect,
the flow rate adjustment means may be switched between a low flow rate mode for relatively reducing the flow rate of gas flowing into the reaction vessel and a high flow rate mode for relatively increasing the flow rate of gas flowing into the reaction vessel, and
the flow rate adjustment means may increase a period of time in the high flow rate mode when the H₂O value detected by the H₂O value detection means is greater than a predetermined value, and may reduce a period of time in the high flow rate mode when the H₂O value detected by the H₂O value detection means is less than a predetermined value.

In the above aspect, the adsorbent may contain amine.

In the above aspect,
the adsorbent may contain polyethylene imine (PEI),
the H₂O value may be a molar ratio of the amount of adsorbed H₂O adsorbed into the adsorbent to an amount of adsorbed CO₂ adsorbed into the adsorbent when an amount of humidification of the adsorbent is zero, and
the predetermined value may be two.

In the above aspect, the adsorbent may contain polyethylene imine (PEI), and may have a honeycomb structure supported on a honeycomb.

In order to achieve the above-described object, one aspect according to the present disclosure is a carbon dioxide recovery method including:
detecting an H₂O value that is a value related to an amount of H₂O adsorbed into an adsorbent, the adsorbent being disposed inside a reaction vessel and configured to adsorb CO₂ and H₂O; and
adjusting a flow rate of gas flowing into the reaction vessel based on the detected H₂O value.

In order to achieve the above-described object, one aspect according to the present disclosure is a carbon dioxide recovery method including:
a low flow rate step of adjusting a flow rate of gas flowing into a reaction vessel in which an adsorbent configured to adsorb CO₂ and H₂O is disposed to a predetermined low flow rate; and
a high flow rate step of setting, after the low flow rate step is performed, the flow rate of gas flowing into the reaction vessel to a higher flow rate than the predetermined low flow rate.

According to the present disclosure, it is possible to provide a carbon dioxide recovery apparatus and a carbon dioxide recovery method by which energy consumption can be reduced so as to improve CO₂ recovery efficiency.

The above and other objects, features and advantages of the present disclosure will become more fully understood from the detailed description given hereinbelow and the accompanying drawings.

### BRIEF DESCRIPTION OF DRAWINGS

FIG. 1 is a block diagram showing a schematic system configuration of a carbon dioxide recovery apparatus according to a present embodiment;

FIG. 2 is a diagram showing an example of a relationship between an integrated amount of adsorbed CO₂ adsorbed by amine in an adsorbent and predetermined times t1 and t2;

FIG. 3 is a diagram for explaining the energy consumption of an air supply apparatus when the air supply apparatus is switched from a low flow rate mode to a high flow rate mode;

FIG. 4 is a diagram showing an example of a relationship between an H₂O value and an amount of adsorbed H₂O adsorbed by amine in an adsorbent; and

FIG. 5 is a diagram showing an example of a relationship between a wind velocity and an amount of adsorbed CO₂ and an amount of adsorbed H₂O.

### DESCRIPTION OF EMBODIMENTS

### First Embodiment

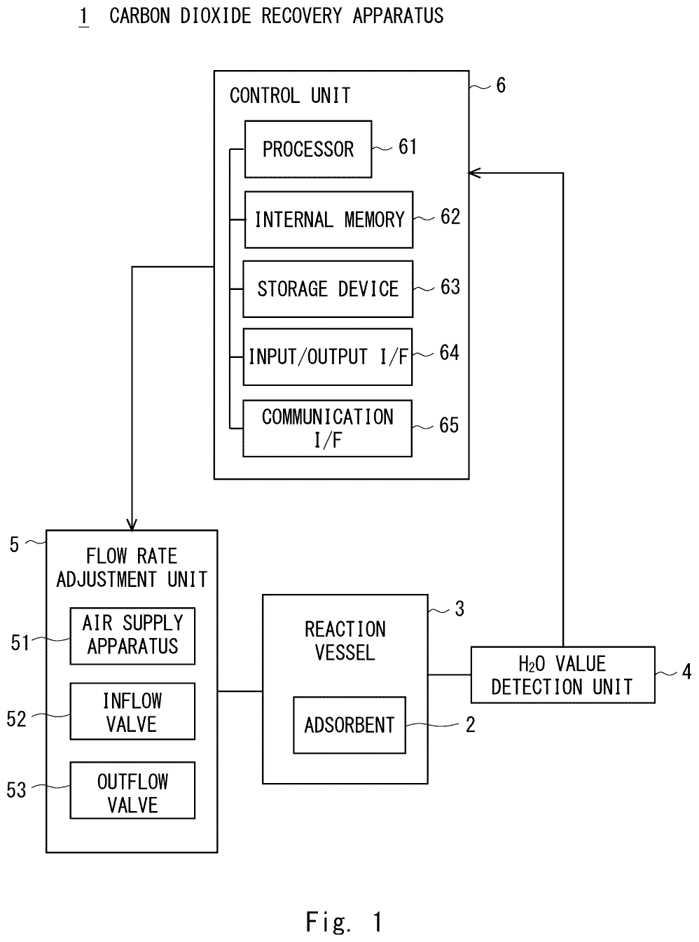

The present embodiment will be described hereinafter with reference to the drawings. FIG. 1 is a block diagram showing a schematic system configuration of a carbon dioxide recovery apparatus according to this embodiment. A carbon dioxide recovery apparatus 1 according to this embodiment is an apparatus for recovering carbon dioxide from the atmosphere by, for example, a Direct Air Capture (DAC) method.

The carbon dioxide recovery apparatus 1 according to this embodiment includes an adsorbent 2, a reaction vessel 3, an H₂O value detection unit 4, a flow rate adjustment unit 5, and a control unit 6.

The adsorbent 2 is configured to adsorb carbon dioxide (CO₂) and H₂O (water) from the surrounding gas. The adsorbent 2 contains, for example, amine (N-(2-aminoethyl)-3-aminopropyl-methyldimethoxysilane, H₂N (CH₂)₂NH (CH₂)₃SiCH₃(OCH₃)₂: AEAPDMS) such as polyethylene imine (PEI). The amine in the adsorbent 2 may be formed as, for example, a fiber filter formed by impregnating the amine into nanofibers (Fibrillated cellulose suspension).

Note that the above configuration of the adsorbent 2 is one example, and is not limited thereto. For example, a method for forming the adsorbent 2 into a form of powder, pellet, or granule, or a method for making the adsorbent 2 supported on a honeycomb may be used. These forms may be determined based on a reaction rate to be required, a pressure loss, purity of CO₂ in the desorbed gas, and the like.

The adsorbent 2 is disposed at a predetermined position in the reaction vessel 3. The reaction vessel 3 is, for example, a sealed box-shaped vessel. Both of an inflow pipe for allowing the outside air to flow into the reaction vessel 3 and an outflow pipe for allowing the inside gas to flow out of the reaction vessel 3 may be connected to the reaction vessel 3. Note that the outside air is not limited to the atmosphere according to the above-mentioned DAC method, and may be, for example, gas discharged from a factory, a power plant, an apparatus, or the like.

The H₂O value detection unit 4 is a specific example of H₂O value detection means. The H₂O value detection unit 4 detects an H₂O value that is a value related to the amount of H₂O adsorbed into the adsorbent 2 in the reaction vessel 3.

The H₂O value is, for example, the amount of adsorbed H₂O adsorbed into the adsorbent 2, the concentration of H₂O, or the like. For example, the H₂O value detection unit 4 detects both the amount of H₂O in the gas flowing into the reaction vessel 3 through the inflow pipe and the amount of H₂O in the gas flowing out of the reaction vessel 3 through the outflow pipe detected by a sensor or the like. Further, the H₂O value detection unit 4 calculates the amount of H₂O adsorbed into the adsorbent 2 based on a difference between the above detected amounts of H₂O.

The flow rate adjustment unit 5 is a specific example of flow rate adjustment means. The flow rate adjustment unit 5 adjusts the flow rate of the gas flowing into the reaction vessel 3 from the inflow pipe based on the H₂O value detected by the H₂O value detection unit 4.

The flow rate adjustment unit 5 is composed of, for example, an air supply apparatus 51 that allows gas to flow into the reaction vessel 3 through the inflow pipe, an inflow valve 52 provided in the inflow pipe and capable of adjusting the inflow amount of gas flowing into the reaction vessel 3, and an outflow valve 53 provided in the outflow pipe and capable of adjusting the outflow amount of gas flowing out of the reaction vessel 3.

The air supply apparatus 51 of the flow rate adjustment unit 5 increases or reduces the flow rate of gas flowing into the reaction vessel 3 through the inflow pipe by increasing or reducing, for example, the wind velocity (output) of a fan in response to a control signal from the control unit 6. The air supply apparatus 51 may have a low flow rate mode for relatively reducing the flow rate of gas flowing into the reaction vessel 3 and a high flow rate mode for relatively increasing the flow rate of the same. The air supply apparatus 51 can adjust the flow rate of gas flowing into the reaction vessel 3 by switching between the low flow rate mode and the high flow rate mode.

Note that the flow rate adjustment unit 5 may adjust the flow rate of gas flowing into the reaction vessel 3 by adjusting at least one of the inflow valve 52 and the outflow valve 53. Further, the air supply apparatus 51 may be provided in the outflow pipe, and may adjust the flow rate of gas flowing into the reaction vessel 3 by adjusting the flow rate of gas flowing out of the reaction vessel 3.

The control unit 6 controls the above-described mode switching performed by the flow rate adjustment unit 5, the opening and closing of the inflow and outflow valves, and the like based on the H₂O value detected by the H₂O value detection unit 4. Note that the control unit 6 and the flow rate adjustment unit 5 may be integrally formed.

The control unit 6 has, for example, a hardware configuration of a normal computer including a processor 61 such as a Central Processing Unit (CPU) or a Graphics Processing Unit (GPU), an internal memory 62 such as a Random Access Memory (RAM) and a Read Only Memory (ROM), a storage device 63 such as a Hard Disk Drive (HDD) or a Solid State Drive (SSD), an input/output I/F 64 for connecting a peripheral device such as a display, and a communication I/F 65 for communicating with a device located outside the data processing apparatus.

The characteristics of the amine in the adsorbent 2 will be described below. Under dry conditions, a stable structure is formed by one molecule of CO₂ being adsorbed into two molecules of amine (AEAPMDS₂). On the other hand, in the presence of H₂O, a stable structure is formed by one molecule of CO₂ being adsorbed into one molecule of amine (AEAPMDS₁). Therefore, the amine in the adsorbent 2 has a characteristic that H₂O has been adsorbed or that the amount of adsorbed CO₂ increases in an environment where there is a large amount of H₂O in the surroundings.

Note that the above characteristic is disclosed in detail in Non-Patent Literature (Cost Evaluation of Direct Air Capture (DAC) Process (Vol. 2): Adsorption Method, Center for Low Carbon Society Strategy, Japan Science and Technology Agency, LCS-FY2020-PP-06), which can be incorporated herein by reference as appropriate.

Further, the amine in the adsorbent 2 has a characteristic that an amount of H₂O absorbed when the flow rate of gas flowing into the reaction vessel 3 is small is larger than an amount of H₂O absorbed when the flow rate of gas flowing into the reaction vessel 3 is large.

In a carbon dioxide recovery method according to this embodiment, the flow rate adjustment unit 5 adjusts the flow rate of gas flowing into the reaction vessel 3 based on the H₂O value detected by the H₂O value detection unit 4 by taking into account the above characteristics of the amine in the adsorbent 2.

According to the carbon dioxide recovery method according to this embodiment, for example, only by adjusting the flow rate of gas flowing into the reaction vessel 3 in accordance with the H₂O value of the adsorbent 2 without using a special H₂O concentration adjustment device or the like, CO₂ in the gas can be adsorbed efficiently into the adsorbent 2 and recovered. Therefore, energy consumption can be reduced so as to improve CO₂ recovery efficiency.

For example, the flow rate adjustment unit 5 first reduces the flow rate of gas flowing into the reaction vessel 3 during a predetermined time t1, thereby causing a relatively large amount of H₂O to be adsorbed into the amine in the adsorbent 2. By doing so, the CO₂ adsorption capacity of the amine in the adsorbent 2 (i.e., the amount of the CO₂ adsorbed into the amine in the adsorbent 2) is temporarily increased by the large amount of H₂O adsorbed into the amine in the adsorbent 2.

Then, the flow rate adjustment unit 5 increases the flow rate of gas flowing into the reaction vessel 3 during a predetermined time t2, thereby supplying a relatively large amount of CO₂ to the amine in the adsorbent 2. By doing so, the supplied large amount of CO₂ can be adsorbed efficiently into the amine in the adsorbent 2 in which the CO₂ adsorption capacity thereof has been increased.

Thus, a large amount of CO₂ can be adsorbed efficiently into the amine in the adsorbent 2 while at the same time the flow rate adjustment unit 5 is efficiently operated to reduce the energy consumption. That is, energy consumption can be reduced so as to improve CO₂ recovery efficiency.

Note that, in this embodiment, although the adsorbent 2 contains amine, the present disclosure is not limited thereto. The adsorbent 2 may instead contain, for example, zeolite or activated carbon having the same characteristics as the above amine.

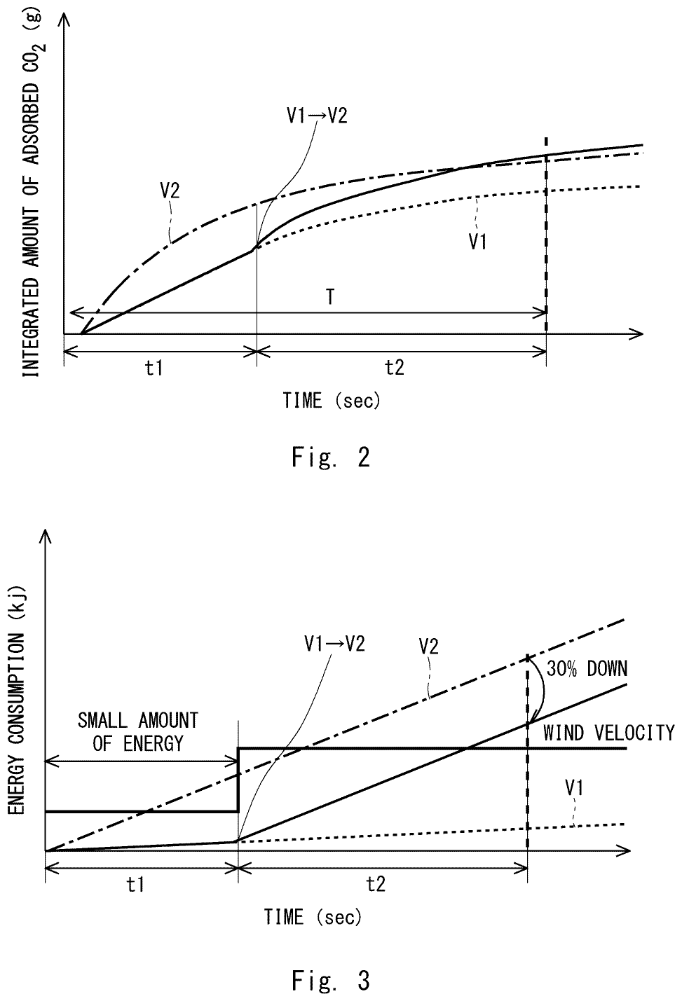

Next, an example of a method for determining the above predetermined times t1 and t2 will be described. FIG. 2 is a diagram showing an example of a relationship between an integrated amount of adsorbed CO₂ adsorbed by amine in the adsorbent and the predetermined times t1 and t2.

In FIG. 2, a dotted line indicates the amount of adsorbed CO₂ adsorbed by the amine in the adsorbent 2 when the outside air is allowed to flow into the reaction vessel 3 at a low wind velocity v1 in the low flow rate mode. A dot-and-dash line indicates the amount of adsorbed CO₂ adsorbed by the amine in the adsorbent 2 when the outside air is allowed to flow into the reaction vessel 3 at a high wind velocity v2 in the high flow rate mode. A solid line indicates the amount of adsorbed CO₂ adsorbed by the amine in the adsorbent 2 when the outside air is allowed to flow into the reaction vessel 3 by switching from the low wind velocity v1 in the low flow rate mode to the high wind velocity v2 in the high flow rate mode as described later.

The air supply apparatus 51 of the flow rate adjustment unit 5 allows the atmosphere having the low wind velocity v1 to flow into the reaction vessel 3 from the inflow pipe for the predetermined time t1 in the low flow rate mode. Then, the air supply apparatus 51 causes the atmosphere having the high wind velocity v2 to flow into the reaction vessel 3 from the inflow pipe for the predetermined time t2 in the high flow rate mode.

First, based on a measured humidity of the atmosphere, an amount H1 of adsorbed H₂O and an amount C1 of adsorbed CO₂ adsorbed by the amine in the adsorbent 2 when gas having the measured humidity is allowed to flow into the reaction vessel 3 at the low wind velocity v1 for the predetermined time t1 are calculated.

After the above amount H1 of adsorbed H₂O is caused to be adsorbed into the amine in the adsorbent 2, the amount C2 of adsorbed CO₂ by the amine in the adsorbent 2 when gas is allowed to flow into the reaction vessel 3 at the high wind velocity v2 for the predetermined time t2 is calculated. The predetermined times t1 and t2 are determined so that the total amount C1+C2 of adsorbed CO₂ during the time T=t1+t2 in all the CO₂ adsorption processes becomes a maximum amount or a certain amount or more. Note that not only the predetermined times t1 and t2 may be changed, but also both of the low wind velocity v1 in the low flow rate mode and the high wind velocity v2 in the high flow rate mode may be changed.

Further, a total energy consumption W1+W2 of the air supply apparatus 51 may be calculated based on an energy consumption W1 of the air supply apparatus 51 in the low flow rate mode during the predetermined time t1 and an energy consumption W2 of the air supply apparatus 51 in the high flow rate mode during the predetermined time t2. Further, the predetermined times t1 and t2 may be determined so that the total energy consumption W1+W2 with respect to the total amount C1+C2 of adsorbed CO₂ is equal to or less than a target value.

FIG. 3 is a diagram for explaining the energy consumption of the air supply apparatus 51 when the air supply apparatus 51 is switched from the low flow rate mode to the high flow rate mode as described above.

In FIG. 3, a dotted line indicates the energy consumption of the air supply apparatus 51 at the low wind velocity v1 in the low flow rate mode. A dot-and-dash line indicates the energy consumption of the air supply apparatus 51 at the high wind velocity v2 in the high flow rate mode. A solid line indicates the energy consumption of the air supply apparatus 51 when the low wind velocity v1 in the low flow rate mode is switched to the high wind velocity v2 in the high flow rate mode. Further, in FIG. 3, for example, v1=1.2 [m/s], v2=3.3 [m/s], t1=2 [m], and t2=3 [m] are set.

As shown in FIG. 3, when the low wind velocity v1 in the low flow rate mode is switched to the high wind velocity v2 in the high flow rate mode, it can be confirmed that the energy consumption can be reduced by approximately 30% with respect to the energy consumption of the air supply apparatus 51 at the high wind velocity v2 in the high flow rate mode.

As described above, in this embodiment, the amount of gas flowing into the reaction vessel 3 by the air supply apparatus 51 is efficiently adjusted by taking into account the CO₂ adsorption capacity corresponding to the amount of adsorbed H₂O adsorbed by the amine in the adsorbent 2. Thus, a large amount of CO₂ can be adsorbed efficiently into the amine in the adsorbent 2 while at the same time the energy consumption of the air supply apparatus 51 is reduced.

That is, energy consumption can be reduced so as to improve CO₂ recovery efficiency.

As described above, any method for desorbing CO₂ from the adsorbent 2 after the adsorbent 2 adsorbs CO₂ may be employed. For example, a temperature swing method in which the temperature of the adsorbent 2 is increased to desorb CO₂ may be used.

The amount of adsorbed H₂O adsorbed by the adsorbent 2 basically continues to increase as gas flows into the reaction vessel 3. However, during the above desorption, the amount of adsorbed H₂O is reset by heating. Therefore, after the resetting, it is necessary to cause H₂O to be adsorbed into the adsorbent 2 as described above in order to cause CO₂ to be adsorbed into the adsorbent 2. Note that examples of a situation in which the amount of adsorbed H₂O decreases may include a case in which the humidity of the atmosphere changes from a high state to a low state and suddenly dry gas flows into the reaction vessel 3.

In the carbon dioxide recovery apparatus 1 according to the above embodiment, first, the atmosphere is allowed to flow into the reaction vessel 3 at the low wind velocity in the low flow rate mode, and then the atmosphere is allowed to flow into the reaction vessel 3 at the high wind velocity in the high flow rate mode.

Meanwhile, in a case of a DAC having a long cycle (one day per cycle), the humidity of the atmosphere may change during that time. For example, it is conceivable that the humidity of the atmosphere may be low in the morning and high at night. In this case, the carbon dioxide recovery apparatus 1 may allow the atmosphere to flow into the reaction vessel 3 at the high wind velocity in the high flow rate mode, and then allow the atmosphere to flow into the reaction vessel 3 at the low wind velocity in the low flow rate mode.

### Second Embodiment

In this embodiment, the flow rate adjustment unit 5 may increase the flow rate of gas flowing into the reaction vessel 3 when the H₂O value detected by the H₂O value detection unit 4 is greater than a predetermined value.

As described above, in a situation where the H₂O value of the amine in the adsorbent 2 is larger than a predetermined value, that is, the CO₂ adsorption capacity of the amine is high, the amount of CO₂ flowing into the reaction vessel 3 is increased. By doing so, CO₂ can be adsorbed efficiently into the amine in the adsorbent 2 as it is not necessary to increase energy consumption.

Meanwhile, when the H₂O value detected by the H₂O value detection unit 4 is less than a predetermined value, the flow rate adjustment unit 5 may reduce the flow rate of gas flowing into the reaction vessel 3.

As described above, in a situation where the H₂O value of the amine in the adsorbent 2 is less than a predetermined value, that is, the CO₂ adsorption capacity of the amine is low, the amount of CO₂ flowing into the reaction vessel 3 is reduced. Thus, by reducing the flow rate of gas flowing into the reaction vessel 3, the amount of adsorbed H₂O adsorbed by the amine in the adsorbent 2 can be increased, and the CO₂ adsorption capacity of the amine can be increased by the increased H₂O.

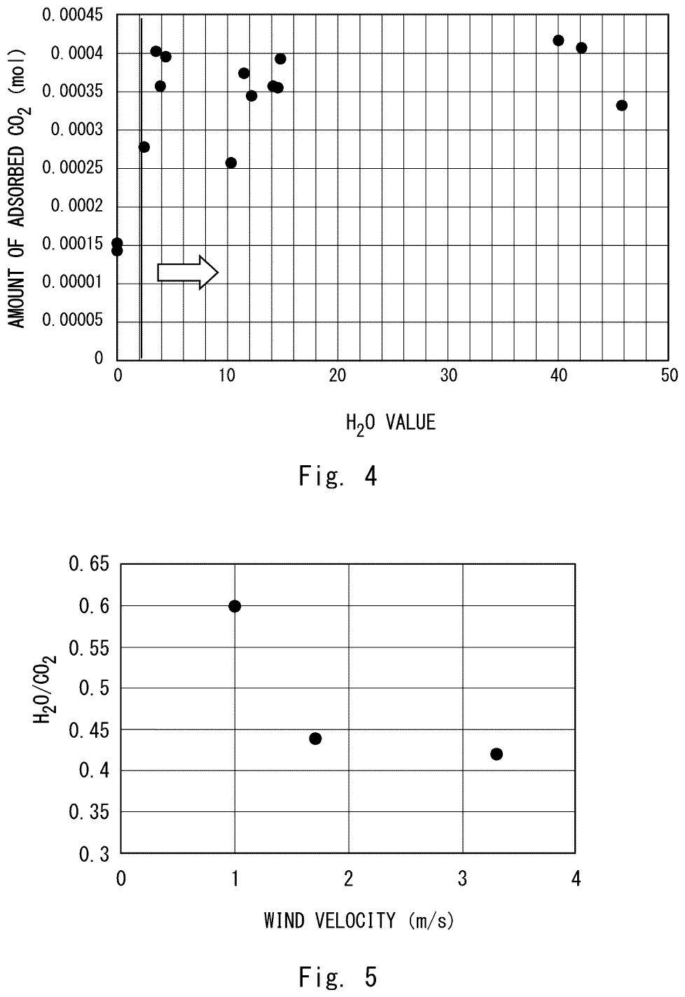

An example of a method for setting the above-described predetermined value will be described below. FIG. 4 is a diagram showing an example of a relationship between the H₂O value and the amount of adsorbed CO₂ adsorbed by the amine in the adsorbent. Note that, in FIG. 4, the H₂O value is, for example, a molar ratio of the amount of adsorbed H₂O adsorbed into the adsorbent 2 to the amount of adsorbed CO₂ adsorbed into the adsorbent 2 when the amount of humidification of the adsorbent 2 is zero. Further, the adsorbent 2 contains polyethylene imine (PEI). As shown in FIG. 4, when the H₂O value exceeds approximately two, the amount of adsorbed CO₂ adsorbed into the adsorbent 2 increases significantly. Therefore, the predetermined value may be set to approximately two.

Further, when the H₂O value detected by the H₂O value detection unit 4 is greater than the predetermined value, the flow rate adjustment unit 5 may increase the predetermined time t2 in the high flow rate mode. Thus, by increasing the predetermined time t2 in the high flow rate mode in which the amount of CO₂ flowing into the reaction vessel 3 increases when the H₂O value (the amount of adsorbed H₂O) is large and the CO₂ adsorption capacity of the amine in the adsorbent 2 is high, CO₂ can be adsorbed efficiently into the adsorbent 2 as it is not necessary to increase energy consumption.

On the other hand, when the H₂O value detected by the H₂O value detection unit 4 is less than the predetermined value, the flow rate adjustment unit 5 may reduce the predetermined time t2 in the high flow rate mode. Thus, by reducing the time in the high flow rate mode in which the amount of CO₂ flowing into the reaction vessel 3 increases when the H₂O value (the amount of adsorbed H₂O) is small and the CO₂ adsorption capacity of the amine in the adsorbent 2 is low, the amount of adsorbed H₂O adsorbed by the amine in the adsorbent 2 can be increased and thus the adsorption capacity of CO₂ can be increased while at the same time the energy consumption is reduced.

### Third Embodiment

In this embodiment, the adsorbent 2 may contain polyethylene imine (PEI), and have a honeycomb structure supported on a honeycomb.

By this structure, as shown in FIG. 5, the amount of adsorbed CO₂ increases by 1.83 times (3.3/1.8) as compared to the case of a normal wind velocity (1.8). Further, the amount of adsorbed H₂O increases by 0.56 times (1.0/1.8) as compared to the case of a normal wind velocity (1.8).

Several novel embodiments according to the present disclosure have been described above. However, these embodiments are merely presented as examples and are not intended to limit the scope of the disclosure. These novel embodiments can be implemented in various forms. Further, their components/structures may be omitted, replaced, or modified without departing from the scope and the spirit of the disclosure. These embodiments and modifications thereof are included in the scope and the spirit of the disclosure and also included in the disclosure specified in the claims and the scope equivalent thereto.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A carbon dioxide recovery apparatus comprising:
an adsorbent configured to adsorb CO₂ and H₂O;
a reaction vessel inside which the adsorbent is disposed;
H₂O value detection means for detecting an H₂O value that is a value related to an amount of H₂O adsorbed into the adsorbent; and
flow rate adjustment means for adjusting a flow rate of gas flowing into the reaction vessel based on the H₂O value detected by the H₂O value detection means.

2. The carbon dioxide recovery apparatus according to claim 1, wherein the flow rate adjustment means increases the flow rate of gas flowing into the reaction vessel when the H₂O value detected by the H₂O value detection means is greater than a predetermined value.

3. The carbon dioxide recovery apparatus according to claim 1, wherein the flow rate adjustment means reduces the flow rate of gas flowing into the reaction vessel when the H₂O value detected by the H₂O value detection means is less than a predetermined value.

4. The carbon dioxide recovery apparatus according to claim 1, wherein
the flow rate adjustment means is switched between a low flow rate mode for relatively reducing the flow rate of gas flowing into the reaction vessel and a high flow rate mode for relatively increasing the flow rate of gas flowing into the reaction vessel, and
the flow rate adjustment means increases a period of time in the high flow rate mode when the H₂O value detected by the H₂O value detection means is greater than a predetermined value, and reduces a period of time in the high flow rate mode when the H₂O value detected by the H₂O value detection means is less than a predetermined value.

5. The carbon dioxide recovery apparatus according to claim 1, wherein the adsorbent contains amine.

6. The carbon dioxide recovery apparatus according to claim 2, wherein
the adsorbent contains polyethylene imine (PEI),
the H₂O value is a molar ratio of the amount of adsorbed H₂O adsorbed into the adsorbent to an amount of adsorbed CO₂ adsorbed into the adsorbent when an amount of humidification of the adsorbent is zero, and
the predetermined value is two.

7. The carbon dioxide recovery apparatus according to claim 1, wherein the adsorbent contains polyethylene imine (PEI), and has a honeycomb structure supported on a honeycomb.

8. A carbon dioxide recovery method comprising:
detecting an H₂O value that is a value related to an amount of H₂O adsorbed into an adsorbent, the adsorbent being disposed inside a reaction vessel and configured to adsorb CO₂ and H₂O; and
adjusting a flow rate of gas flowing into the reaction vessel based on the detected H₂O value.

9. A carbon dioxide recovery method comprising:
a low flow rate step of adjusting a flow rate of gas flowing into a reaction vessel in which an adsorbent configured to adsorb CO₂ and H₂O is disposed to a predetermined low flow rate; and
a high flow rate step of setting, after the low flow rate step is performed, the flow rate of gas flowing into the reaction vessel to a higher flow rate than the predetermined low flow rate.
